SEQUENCE LISTING (7 sequences)

SEQ ID NO: 1 - DNA, 20 bp, Clostridium perfringens
ttacctggagtggctccaac

SEQ ID NO: 2 - DNA, 20 bp, Clostridium perfringens
acacctggtccttgagcatc

SEQ ID NO: 3 - DNA, 19 bp, Clostridium perfringens
agtgtaattagtacaagcc

SEQ ID NO: 4 - DNA, 21 bp, Clostridium perfringens
ggccatttcatttttccgtaa

SEQ ID NO: 5 - DNA, 26 bp, Clostridium perfringens
tctcttaaagattttggttcctctgg

SEQ ID NO: 6 - DNA, 25 bp, Clostridium perfringens
acattatttgctgcattaacaacag

SEQ ID NO: 7 - DNA, 283 bp, Clostridium perfringens
atgatgcaaattttagcatcatgggatataaaatttgttgagactaaggacggttataatatagattcttatcatgctatttatggaaatcaattattcatgaaatcaagattgtataataatggtgataaaaatttcacagatgatagagatttatcaacattaatttctggtggattttcacccaatatggctttagcattaacagcacctaaaaatgctaaagaatctgtaataatagttgaatatcaaagatttgataatgactatattttaaattggg

(19) United States
(12) Patent Application Publication
(10) Pub. No.: US 2021/0283199 A1
(43) Pub. Date: Sep. 16, 2021

(54) METHOD FOR CONTROLLING CLOSTRIDIUM INFECTION IN ANIMALS

(71) Applicant: KEMIN INDUSTRIES, INC., Des Moines, IA (US)

(72) Inventors: Pei Wern LIM, Singapore (SG); BoonFei TAN, Singapore (SG); JXi NG, Singapore (SG); Hai Meng TAN, Singapore (SG); Chuan Hao TAN, Singapore (SG)

(21) Appl. No.: 17/200,811

(22) Filed: Mar. 13, 2021

Related U.S. Application Data
(60) Provisional application No. 62/989,398, filed on Mar. 13, 2020.

(51) Int. Cl. A61K 35/742; C07K 14/32

(52) U.S. Cl. CPC A61K 35/742; A61K 38/00; C07K 14/32

(57) ABSTRACT

The present invention relates to compositions and methods of inhibiting quorum sensing processes of C. perfringens using fengycin. Fengycin effectively downregulates the expression of agrD and netB genes, resulting in reduced pathogenesis of C. perfringens. The composition may contain fengycin or a source of fengycin, such as Bacillus subtilis PB6.

### CROSS-REFERENCE TO RELATED APPLICATIONS

This application claims the benefit of priority to U.S. Provisional Patent Application No. 62/989,398, filed Mar. 13, 2020, entitled “A METHOD FOR CONTROLLING CLOSTRIDIUM INFECTION IN ANIMALS,” the entire disclosure of which is incorporated by reference in its entirety.

### BACKGROUND OF THE INVENTION

Quorum sensing (QS) is a cell-cell communication system that bacteria use to coordinate social behavior in response to changes in population density. QS regulates the pathogenicity of many animal and human pathogens including Gram-positive Clostridium perfringens and Staphylococcus aureus (1), as well as Gram-negative Escherichia coli (2) and Salmonella spp. (3). QS process involves the production, secretion and detection of diffusible signaling molecules or autoinducers (AI) in a cell density-dependent manner. When reaching a signal threshold, the AI bind to the receptors on the cell surface or in the cytoplasm of pathogens and trigger a cellular response including production of toxins and other virulence factors required, for example, for gut colonization.

C. perfringens is an etiological agent for avian necrotic enteritis. In C. perfringens, the biosynthesis of necrotizing toxin, including netB toxin responsible for avian necrotic enteritis, is under the regulation of the agr-type QS system (4-9). Necrotic enteritis is a disease condition affecting the intestine. Under certain conditions, C. perfringens can proliferate to high number and produce extracellular toxins, causing intestinal lesions which often lead to high mortality rate. The agr-type QS system in C. perfringens regulates the production of signaling molecule termed the autoinducer peptide (AIP) (4). Individual C. perfringens cells produce and secrete AIP into the environment, and once the AIP accumulates to a certain concentration threshold, these molecules will interact with a membrane receptor and trigger a collective response of the C. perfringens population in expressing many virulence factors including toxin production, colonization factors and biofilm formation, all of which are important in infection and disease progression (4).

Conventionally, C. perfringens infection is treated using antibiotic. Antibiotic, however, will not be administered to healthy or non-symptomatic animals. Antibiotics are also used as growth promoters in livestock in certain countries. The use of antibiotic as growth promoter to manage pathogens that can potentially cause diseases in livestock is discouraged in general as such practices encourage the proliferation of antibiotic-resistant pathogens.

Alternatively, Bacillus subtilis PB6 can also be used as a probiotic to manage C. perfringens infection due to its ability of secreting surfactin-a well-known lipopeptide that can penetrate cell membrane of C. perfringens, causing cytoplasm leakage and ultimately cell death. The function of B. subtilis PB6 in killing C. perfringens and other pathogenic bacteria by way of producing antimicrobial peptides has been previously disclosed in the applicant's prior U.S. Pat. No. 7,247,299, the entire disclosure of which is hereby incorporated by reference. This patent, however, only focused upon the ability of B. subtilis PB6 in producing antimicrobial peptides as a bactericidal agent.

Fengycin is a cyclic lipopeptide with anti-fungal activities naturally produced by B. subtilis PB6. The effect of fengycin on C. perfringens has not been reported in the literature thus far. The present invention relates to the discovery that fengycin is able to inhibit quorum sensing signaling by C. perfringens. This new mode of action can be used to more specifically target diseases and manage diseases caused by C. perfringens by way of disrupting quorum formation required for pathogenicity.

### SUMMARY OF THE INVENTION

The present invention relates to methods of using fengycin produced by B. subtilis PB6 for management and treatment of C. perfringens infection. The B. subtilis PB6 can be used to disrupt QS required for the expression of virulence factors including toxin production and biofilm formation. The inventors have determined that fengycin produced by B. subtilis PB6 downregulates the expression of agrD gene, which is involved in the formation of the autoinducer peptide required to trigger toxin production. The fengycin or fengycin-containing B. subtilis PB6 spent media may be used in the treatment of avian necrotic enteritis or other diseases under the regulation of the agr-type QS system.

### DETAILED DESCRIPTION OF THE FIGURES

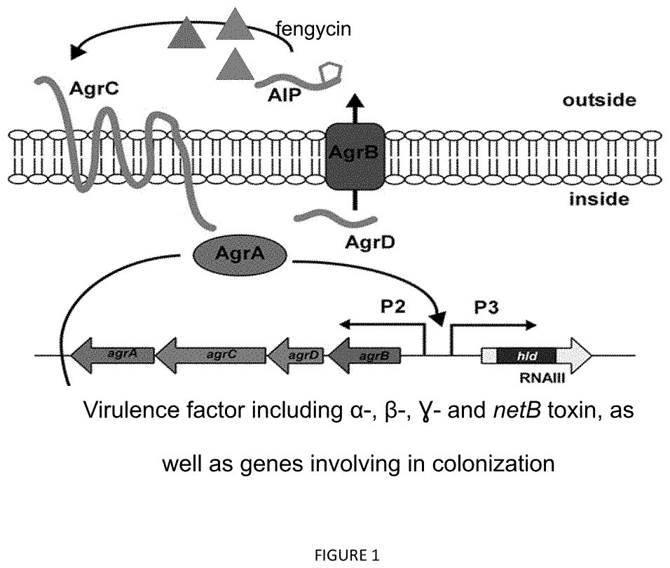

FIG. 1 illustrates QS system of gram positive bacteria and how fengycin may interrupt QS signalling through competitive inhibition of the cognate AIP from binding to the trans-membrane AgrC receptor.

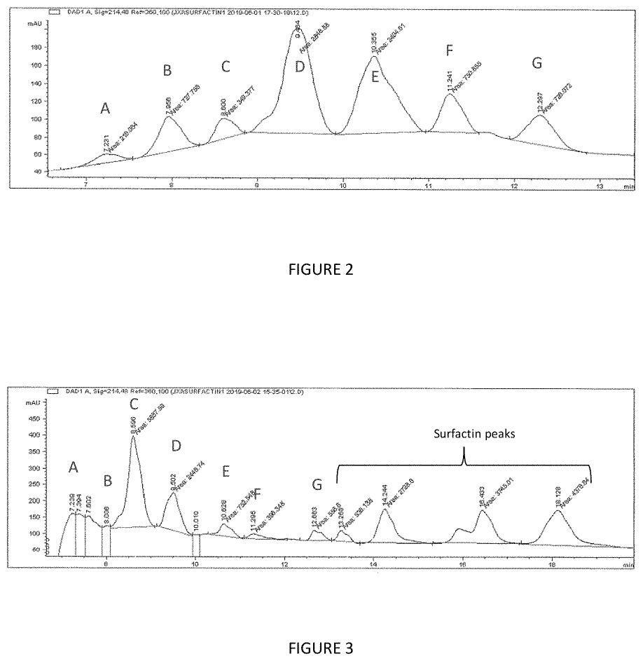

FIG. 2 illustrates an HPLC chromatogram of standard fengycin obtained from Sigma Aldrich as described in Example 1. The final concentration of fengycin is equivalent to the sum of area under curve of peaks A, B, C, D, E, F and G.

FIG. 3 illustrates an HPLC chromatogram of fengycin extracted from B. subtilis PB6 overnight culture supernatant as set forth in Example 1. The final concentration of fengycin is equal to the sum of peaks A, B, C, D, E, F and G.

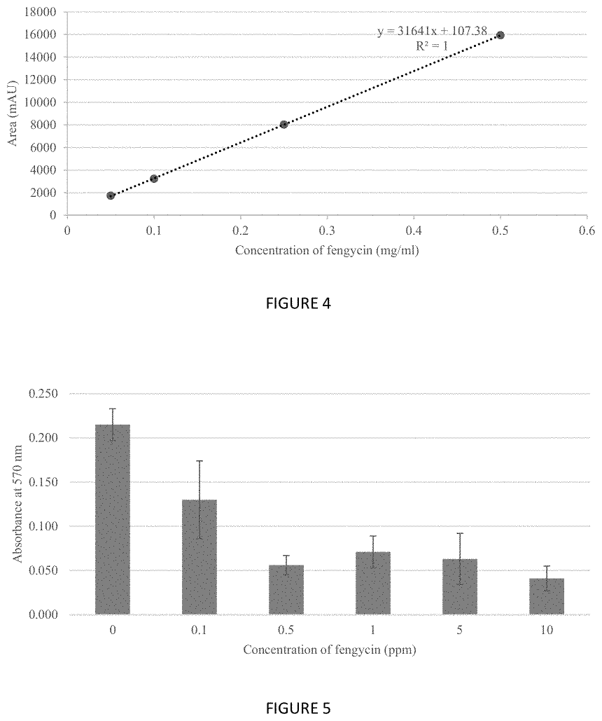

FIG. 4 illustrates the standard calibration curve for pure fengycin from Sigma Aldrich as described in Example 1. B. subtilis PB6 produces 3.2 ppm fengycin when cultured in TSBYE broth overnight at 37° C.

FIG. 5 is a bar graph illustrating biofilm formation by wild-type C. perfringens strain 4.6 in culture added with 0 to 10 ppm fengycin as described in Example 1. Each bar represents an average of 3 replicates (n=3).

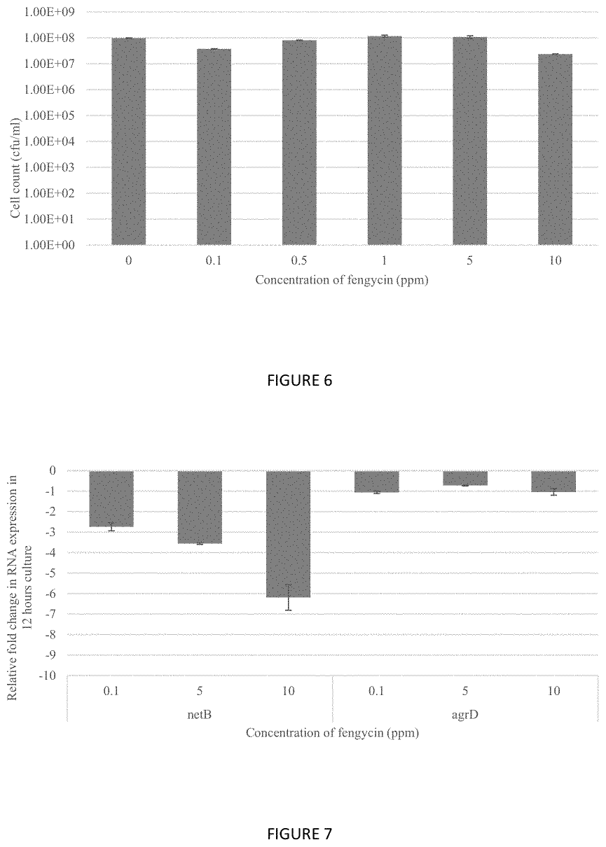

FIG. 6 is a bar graph illustrating cell count of wild-type C. perfringens strain 4.6 (cfu/ml) in culture broth added with 0 to 10 ppm of fengycin as described in Example 1. Each bar represents an average of 3 replicates (n=3).

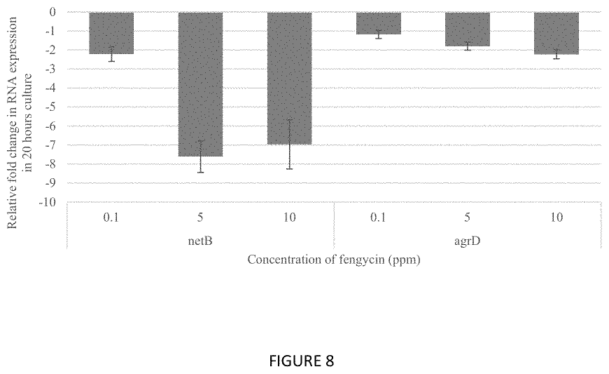

FIG. 7 is a bar graph illustrating relative expression of agrD and netB genes by C. perfringens strain 4.6 after being cultured for 12 hours in the presence of 0.1, 5 and 10 ppm of fengycin. The relative expression is in comparison to the untreated control.

FIG. 8 is a bar graph illustrating relative expression of agrD and netB genes by C. perfringens strain 4.6 after being cultured for 20 hours in the presence of 0.1, 5 and 10 ppm of fengycin. The relative expression is in comparison to the untreated control.

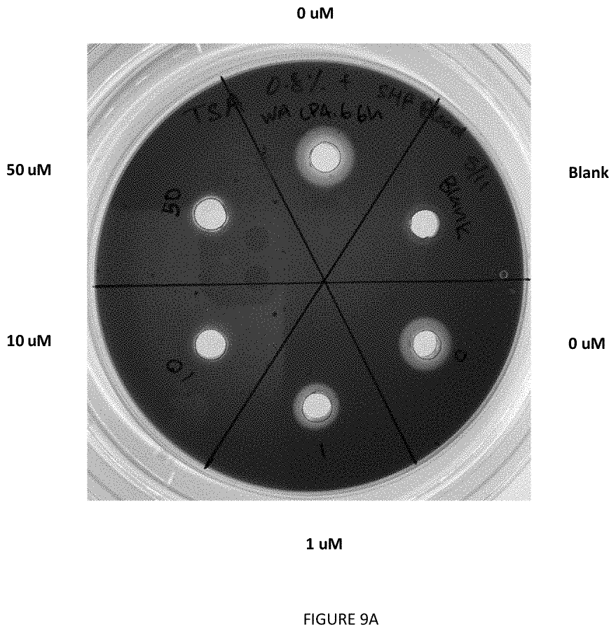

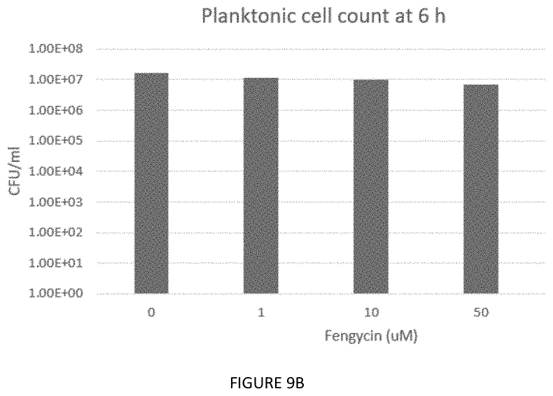

FIG. 9A is a photograph showing the hemolysis mediated by perfringolysin O (pfoA) toxin secreted by C. perfringens CP4.6 as described in Example 2.

FIG. 9B is a graph showing planktonic cell count for C. perfringens CP4.6 exposing to different concentrations of fengycin after incubation for 6 h at 37° C., as described in Example 2.

### DETAILED DESCRIPTION OF THE PREFERRED EMBODIMENT

The present invention relates to methods and compositions for using B. subtilis PB6 to manage infections caused by C. perfringens by way of disrupting quorum sensing of the pathogen.

B. subtilis PB6 produces fengycin, a lipopeptide naturally during growth. The present inventors have discovered that fengycin, at concentrations as low as 0.1 ppm, downregulates the expression of agrD (involved in the production of an autoinducer peptide) and netB genes (production of necrotic enteritis toxin) in wild type C. perfringens.

FIG. 1 is an illustration of how the invention works, whereby the precursor (AgrD) of autoinducer peptide (AIP) is encoded by the agrD gene. Once the agrD is formed, it is processed by the agrB protein, and secreted into the environment as AIP. This AIP interacts with the agrC receptor protein, and causes a cascading effect, resulting in downstream activation of virulence factors. It is hypothesized that fengycin competes for binding site with AIP and prevents the overproduction of virulence genes, resulting in reduced pathogenesis of C. perfringens.

The present invention may be used to treat diseases or conditions controlled by QS, for instance diseases or conditions controlled by QS caused by pathogenic C. perfringens. C. perfringens is a gram-positive spore-forming anaerobic bacteria that is normally found in the intestines of humans and animals. It is a common cause of food poisoning when ingested in sufficient numbers and is known to cause infections of the skin and deeper tissues as well several other disorders, including gastroenteritis. In one embodiment, the invention is used in the treatment of avian necrotic enteritis.

According to at least one embodiment of the invention, chickens or other animals are treated with and/or fed a composition containing one or more forms or sources of fengycin. Any form or source of this compound is suitable for this purpose, and may include, but is not limited to, B. subtilis PB6 spores and its culture supernatant. The concentration of fengycin in B. subtilis PB6 overnight culture (tryptone soy broth supplemented with 0.6% yeast) can be as high as 3 ppm. Fengycin, at concentration as low as 0.1 ppm, inhibits quorum sensing processes of C. perfringens by downregulating the expression of agrD and netB genes.

The feed compositions of the invention should be included or administered from at least about 0.001 grams of fengycin/ton of feed, with between about 0.005 to 0.100 grams fengycin/ton of feed, or 10¹¹ colony-forming unit/ton of feed known to produce fengycin (in at least one embodiment, B. subtilis PB6) feed. According to at least one embodiment, the feed composition further comprises B. subtilis and most preferably PB6, which is commercially available as CLOSTAT (Kemin Industries, Inc., Des Moines, Iowa).

The compositions of the invention can further include other ingredients or compounds that may be beneficial for the animal or human to which they are administered including, but not limited to, antioxidants, carbohydrate, protein, fat and oil, vitamins, minerals, probiotics, medicines, flavors, colors, etc. The compositions may also be combined with a pharmaceutically acceptable carrier that may include one or more carriers or excipients, such as fillers, diluents, binders, lubricants, and disintegrants. Such ingredients and their relative amounts to be included are well known to persons skilled in the art.

While the compositions of the invention are described in particular for administration in the animal's feed, the compositions may likewise be administered in the animal's water source. In addition, the compositions may be administered via conventional pharmaceutical routes including, but not limited to, intravenously, rectally, sublingually, etc. via a pharmaceutical carrier appropriate to the selected route of administration.

The ingredients of the formulation may be combined by simply mixing at room temperature (25-30° C.) with agitation. The ingredients of the invention can either be mixed sequentially or can be added all at once to achieve the unique composition of the invention. In preferred embodiments the ingredients are mixed with agitation to improve miscibility. The ingredients can also be mixed without agitation. The composition can in turn be simply combined with the animal feed prior to administration to the animals.

Once combined with the animal feed, the composition of the invention is preferably provided to the animals ad libitum, and preferably for a time period of seven days or more and optimally through the life of the animal. If the composition is administered by a route other than via the animal's feed, the composition is administered in the appropriate vehicle for the particular route over a time period of seven days or more in an amount ranging of from about 0.1-100 μg fengycin/day.

The following examples are offered to illustrate but not limit the invention. Thus, they are presented with the understanding that various formulation modifications as well as method of delivery modifications may be made and still are within the spirit of the invention.

### Example 1

### Inhibition of C. perfringens Biofilm Formation by Fengycin

### Materials and Methods

Strain, culture media and chemical. Wild-type C. perfringens strain 4.6 was isolated from a farm in Japan. TGY broth (3% tryptone soya broth (Oxoid), 2% glucose, 1% yeast extract (Oxoid) and 0.1% sodium thioglycolate (Sigma Aldrich) was used to culture C. perfringens strain 4.6 (10). Pure fengycin (≥90%) used in this study was purchased from Sigma Aldrich.

Detection and quantification of fengycin in culture supernatant of B. subtilis P86. B. subtilis PB6 was revived by streaking on Tryptone Soya Agar with 0.6% yeast extract (TSAYE) and incubated at 37° C. for 18 hours. After 18 hours, a single colony of B. subtilis PB6 was cultured in 25 mL of Tryptone Soya Broth with 0.6% yeast extract (TSBYE) in a 50 mL conical tube. The tube was loosely capped and incubated at 37° C. for 18 hours with shaking at 150 rpm. The overnight culture broth was centrifuged at 4000 rpm for 10 mins. 5×10 mL of supernatant was collected and transferred to 5×50 mL conical tube containing 5×100 mg of conditioned resin Diaion HP-20 (Supelco). After incubation at room temperature 25° C. for 6 hours with shaking at 200 rpm, the supernatant was discarded. The resin containing the bound fengycin variant was washed twice with 10 mL of distilled water/phosphate buffered saline (PBS) and twice with 10 ml of 50% HPLC grade methanol (v/v). Fengycin variants were eluted from the resin with 1 mL of 100% HPLC grade methanol. The eluates were dried down and resuspend in 250 μl of 50% HPLC grade methanol (v/v) (11). Sample was then analyzed using published Reverse Phase-HPLC method with slight modification (12).

Detection and quantification of fengycin in B. subtilis PB6 supernatant using Reverse Phase-HPLC method. This HPLC method on the detection and quantification of fengycin was adopted from a published method (12) with slight modification. The Reverse Phase-HPLC system used was a LC 1100 (Agilent Technologies, Singapore) with an Atlantis dC-18 column of dimensions 4.6 mm×150 mm column and particle size 5 μm (Waters, USA). The flow rate of the mobile phase was 1.1 mL/min with the initial gradient starting from 50 to 80% acetonitrile in 0.1% trifluoroacetic acid in the first 15 mins. The gradient remained at 80% for 20 mins before increasing to 100% for 5 mins as a washing step, returning to 50% once again. A 50 ul sample was injected into each run which lasted 60 mins and eluent absorbance was monitored at 214 nm. The areas of peaks (sample) between 7 to 12.5 mins which were identified as having the same retention times as those peaks from the Sigma standard were added to give the total fengycin peak area (12). Identification of C. perfringens strain 4.6 using biochemical analysis and sanger sequencing. Identification of C. perfringens strain 4.6 was previously carried out using various biochemical analyses including iron milk test, gelatin liquefaction test, nitrate reduction test and motility test, reported internally (data not shown). PCR of the netB gene was performed using primers listed in Table 1, and the PCR product was sequenced using a Sanger sequencing reaction. Effect of fengycin on C. perfringens strain 4.6 biofilm formation. Biofilm formation by C. perfringens strain 4.6 was assessed in Nunc 24-well polystyrene plates (Nunclon™ Delta). C. perfringens strain 4.6 was revived by streaking on Perfringens Agar (OPSP, OXOID) without Supplement A (SR0076, Sodium sulphadiazine) and B (SR0077, Oleandomycin phosphate and Polymyxin B) and incubated anaerobically at 37° C. for 18 hours. Few C. perfringens strain 4.6 colonies were selected and resuspended in 10 mL of TGY broth to achieve cell density of 10⁸ cfu/mL. C. perfringens strain 4.6 was cultured in 2 mL of TGY broth in Nunc 24-well polystyrene plates (Nunclon™ Delta) incubated at 37° C. without shaking with an initial cell density of 10⁶ cfu/mL. Fengycin (5000 ppm stock in MeOH) was added (100 uL) to final concentrations of 0.1, 0.5, 1, 5 and 10 ppm. TGY broth with MeOH and no fengycin was used as control. The plate was incubated at 37° C. anaerobically without shaking for 72 hours. After 72 hours of incubation, the culture liquid was collected, and plate count was carried out using OPSP agar without Supplement A and B. Individual wells were then washed twice with phosphate buffered saline (PBS) pH 7.4 and dried for 20 mins. Then, 1 mL of 0.2% crystal violet solution in 96% ethanol was added into each well and incubated for 20 mins. Next, the crystal violet solution was removed, and the wells were washed three times with PBS. After 30 mins of air drying, 1 mL of 96% ethanol was added to resolubilize bound crystal violet and the absorbance was read at 570 nm using the multimode microplate reader Varioskan™ LUX (13).

Reverse Transcriptase-Quantitative PCR (QPCR). C perfringens strain 4.6 was revived by streaking on Perfringens Agar (OPSP) without Supplement A and B and incubated anaerobically at 37° C. for 18 hours. One hundred and thirty-five microliters of C. perfringens at 10⁶ cfu/mL was inoculated into 150 uL TGY broth per well using a 96-well tissue culture plate (TPP). Fengycin (5000 ppm stock in MeOH) was added (15 uL) to final concentrations of 0.1, 5 and 10 ppm. TGY broth with MeOH but without fengycin was used as control. The plate was incubated anaerobically at 37° C. without shaking for 12 and 20 hours. RNA extraction was carried out using the QIAGEN RNeasy Kit (Qiagen, USA) as per manufacturer's instructions. Complimentary DNA (cDNA) was generated using a cDNA RT Kit (Invitrogen, USA). The reaction mixture contains 4 μL of RNA template and 16 μl of RT-master mix (RT random hexamer primers, dNTP mix, Nuclease-free water, 5×SSIV buffer, 100 mM DTT, RNaseOUT RNA inhibitor and Superscript IV RT), with the following incubation conditions: 23° C. for 10 mins, 55° C. for 10 mins and 80° C. for 10 mins in a standard PCR machine. Finally, 1 μL of cDNA was assayed using StepOne Real-Time PCR System with FastStart Universal SYBR Green Master (ROX) and primers listed in Table 1. Relative expression of target genes was quantified using the comparative C_{T} method (2^{-ΔΔCT}) with rpoB gene as a reference.

TABLE 1
Real time PCR primers used in this study to
quantify the relative expression of target genes

| Primary | | |
|---|---|---|
| name | Primer sequence | Citation |
| rpoA-F1 | TTACCTGGAGTGGCTCCAAC | (4) |
| | (SEQ ID NO. 1) | |
| rpoA-R1 | ACACCTGGTCCTTGAGCATC | (4) |
| | (SEQ ID NO. 2) | |
| netB-F | AGTGTAATTAGTACAAGCC | (4) |
| | (SEQ ID NO. 3) | |
| netB-R | GGCCATTTCATTTTTCCGTAA | (4) |
| | (SEQ ID NO. 4) | |
| agrD-8F | TCTCTTAAAGATTTTGGTTCCTCTGG | Designed in |
| | (SEQ ID NO. 5) | this study |
| agrD-108R | ACATTATTTGCTGCATTAACAACAGT | Designed in |
| | (SEQ ID NO. 6) | this study |

Primers agrD-8F and agrD-108R were designed using Geneious Prime (11) based on the conserved region of the AgrD gene present in Clostridium perfringens

### Results

Detection and Quantification of Fengycin in Culture Supernatant of Bacillus subtilis PB6

FIG. 2 illustrates the HPLC chromatogram of the pure fengycin while FIG. 3 illustrates the HPLC chromatogram of extracted fengycin from Bacillus subtilis PB6 overnight culture supernatant, with the final concentration of fengycin is equal to the sum of peaks under curve of A, B, C, D, E, F, and G. FIG. 4 illustrates standard curve of fengycin standard from 50 to 500 ppm. B. subtilis PB6 produces 3.2 ppm fengycin when cultured in TSBYE broth overnight at 37° C.

NetB Gene Sequence of C. perfringens Strain 4.6

The C. perfringens isolate used in this study has been previously isolated from a poultry farm in Japan. Biochemical typing (14) and sequencing of the netB gene (>99.7% nucleotide sequence identity to multiple C. perfringens strains) confirm the isolate is genetically capable of causing necrotic enteritis based on the presence of netB gene.

NetB sequence obtained from strain 4.6
(SEQ ID NO. 7)
ATGATGCAAATTTTAGCATCATGGGATATAAAATTTGTTGAGACTAAGGA
CGGTTATAATATAGATTCTTATCATGCTATTTATGGAAATCAATTATTCA
TGAAATCAAGATTGTATAATAATGGTGATAAAAATTTCACAGATGATAGA
GATTTATCAACATTAATTTCTGGTGGATTTTCACCCAATATGGCTTTAGC
ATTAACAGCACCTAAAAATGCTAAAGAATCTGTAATAATAGTTGAATATC
AAAGATTTGATAATGACTATATTTTAAATTGGG

### Biofilm Staining and Cell Count

C. perfringens strain 4.6 was cultured in the presence of 0.1, 0.5, 1, 5 and 10 ppm of fengycin and incubated for 72 hours anaerobically without shaking. Fengycin at all tested concentrations inhibited the ability of the organism to form biofilm (FIG. 5). However, at the same concentration range, fengycin did not interfere and had minimal effect on cell growth (FIG. 6).
Reverse Transcriptase-Quantitative PCR (qRT-PCR)
C. perfringens strain 4.6 was cultured in the presence of 0.1, 5 and 10 ppm of fengycin and incubated for 12 and 20 hours anaerobically without shaking. The expression of netB and agrD genes were downregulated when C. perfringens strain 4.6 was exposed to fengycin at 0.1 to 10 ppm, as shown in FIGS. 7 and 8.

### Example 2

### Fengycin Inhibition of Perfringolycin O Expression by C. perfringens

### Materials and Methods

A single colony of C. perfringens strain 4.6 (CP4.6) was inoculated in TGYST (tryptone glucose yeast supplemented with sodium thioglycolate) medium for 16 h. The bacterial culture was diluted 100× in fresh TGYST medium supplemented with fengycin (Sigma Aldrich) and incubated anaerobically at 37° C. for 6 h. A negative solvent control was included in the experiment. Total cell count was determined by serial dilution in TGYST and plated on TSA (tryptic soy agar) at the end of experiment. Cell-free supernatant was obtained by centrifugation at 14,000 rpm for 5 min and filtration via a 0.2 um filter (CA filter, Sartorius). A total of 75 ul cell-free supernatant was dispended to the hole created on the TSA supplemented by 5% sheep blood (Thermo Scientific). The plate was incubated anaerobically at 37° C. for 24 h prior to imaging.

### Results

C. perfringens strain 4.6 (CP4.6) is a Type G farm isolate that produces and secretes PFO to the environment. The presence of PFO in the culture medium can be determined semi-quantitatively using the hemolysis bioassay. PFO breaks down the red blood cells and hemoglobin completely and leaves a clear zone on blood agar. In this study, the impact of fengycin on PFO production by CP4.6 was determined. The data showed that fengycin inhibited pfoA gene expression in a concentration-dependent manner (FIG. 9A). The hemolysis activity (i.e., zone of clearing) was reduced significantly by 1 μM fengycin and completely inhibited by 10 μM and 50 μM fengycin as compared to the negative solvent control (i.e., 0 μM fengycin). Importantly, at the same concentration range, fengycin did not interfere and had minimal effect on C. perfringens cell growth (FIG. 9B).

### CONCLUSION

Fengycin inhibits perfringolycin O expression by C. perfringens in a concentration-dependent manner without compromising the bacterial cell growth.

It should be appreciated that minor dosage and formulation modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplishes at least all of the intended objectives.

### REFERENCES

1. Piewngam P, Zheng Y, Nguyen T H, Dickey S W, Joo H-S, Villaruz A E, Glose K A, Fisher E L, Hunt R L, Li B, Chiou J, Pharkjaksu S, Khongthong S, Cheung G Y C, Kiratisin P, Otto M. 2018. Pathogen elimination by probiotic Bacillus via signalling interference. Nature 562:532-537.
2. Park H, Lee K, Yeo S, Shin H, Holzapfel W H. 2017. Autoinducer-2 Quorum Sensing Influences Viability of Escherichia coli 0157:H7 under Osmotic and In Vitro Gastrointestinal Stress Conditions. Front. Microbiol. 8:1077.
3. Choi J, Shin D, Ryu S. 2007. Implication of quorum sensing in Salmonella enterica serovar typhimurium virulence: the luxS gene is necessary for expression of genes in pathogenicity island
1. Infect. Immun. 75:4885-90.
4. Yu Q, Lepp D, Mehdizadeh Gohari I, Wu T, Zhou H, Yin X, Yu H, Prescott J F, Nie S-P, Xie M-Y, Gong J. 2017. The Agr-Like Quorum Sensing System Is Required for Pathogenesis of Necrotic Enteritis Caused by Clostridium perfringens in Poultry. Infect. Immun. 85.
5. Keyburn A L, Boyce J D, Vaz P, Bannam T L, Ford M E, Parker D, Di Rubbo A, Rood J I, Moore R J. 2008. NetB, a new toxin that is associated with avian necrotic enteritis caused by Clostridium perfringens. PLoS Pathog 4:e26.
6. Chen J, McClane B A. 2012. Role of the Agr-like quorum-sensing system in regulating toxin production by Clostridium perfringens type B strains CN1793 and CN1795. Infect. Immun. 80:3008-17.
7. Vidal J E, Ma M, Saputo J, Garcia J, Uzal F A, McClane B A. 2012. Evidence that the Agr-like quorum sensing system regulates the toxin production, cytotoxicity and pathogenicity of Clostridium perfringens type C isolate CN3685. Mol. Microbiol. 83:179-194.
8. Ohtani K, Yuan Y, Hassan S, Wang R, Wang Y, Shimizu T. 2009. Virulence gene regulation by the agr system in Clostridium perfringens. J. Bacteriol. 191:3919-27.
9. Chen J, Ma M, Uzal F A, McClane B A. 2014. Host cell-induced signaling causes Clostridium perfringens to upregulate production of toxins important for intestinal infections. Gut Microbes 5:96-107.
10. Vidal J E, Shak J R, Canizalez-Roman A. 2015. The CpAL quorum sensing system regulates production of hemolysins CPA and PFO to build Clostridium perfringens biofilms. Infect lmmun 83:2430-2442. Doi.10.1128/IA1.00240-15.
11 Appleyard A N, Choi Shaila, Read D M, Lightfoot A, Boakes S, Hoffman A, Chopra 1, Bierbaum G, Rudd B A M, Dawson M J, Cortes J. 2009. Dissecting Structural and Functional Diversity of the Lantibiotic Mersacidin. Chemistry & Biology. 16: 490-498.
12. De Andrade C J, F C Barros F, De Andrade L M, Rocco S A, Sforca M L, Pastore G M, Jauregi P. 2015. Ultrafiltration based purification strategies for surfactin produced by Bacillus subtilis LBSA using cassava wastewater as substrate. JChemTechnolBiotechnol. 91:3018-3027
13. Kayumov A R, Nureeva A A, Trizna E Y, Gazizova G R, Bogachv M I, Shtyrlin, N V, Pugachev M V, Sapozhnikov S V, Shtyrlin Y G. 2015. New derivatives of pyridoxine exhibit high antibacterial activity against biofilm-embedded Staphylococcus cells. Biomed Research Int. http://dx/doi.org/10.1155/2015/890968.
14. Kearse M, Moir R, Wilson A, Stones-Havas S, Cheung M, Sturrock S, Buxton S, Cooper A, Markowitz S, Duran C, Thierer T, Ashton B, Meintjes P, Drummond A. 2012. Geneious Basic: an integrated and extendable desktop software platform for the organization and analysis of sequence data. Bioinformatics 28:1647-9.
15. Ohtani, K., Yuan, Y., Hassan, S., Wang, R., Wang, Y., & Shimizu, T. (2009). Virulence gene regulation by the agr system in Clostridium perfringens. Journal of bacteriology, 191(12), 3919-927.
16. Cheung, J. K., Keyburn, A. L., Carter, G. P., Lanckriet, A. L., Van Immerseel, F., Moore, R. J., & Rood, J. 1. (2010). The VirSR two-component signal transduction system regulates NetB toxin production in Clostridium perfringens. Infection and immunity, 78(7), 3064-3072.
17. Li, J., Chen, J., Vidal, J. E., & McClane, B. A. (2011). The Agr-like quorum-sensing system regulates sporulation and production of enterotoxin and beta2 toxin by Clostridium perfringens type A non-food-borne human gastrointestinal disease strain F5603. Infection and immunity, 79(6), 2451-2459.
18. Chen, J., & McClane, B. A. (2012). Role of the Agr-like quorum-sensing system in regulating toxin production by Clostridium perfringens type B strains CN1793 and CN1795. Infection and immunity, 80(9), 3008-3017,
19. Harris, R. W., Sims, P. J., & Tweten, R. K. (1991). Kinetic aspects of the aggregation of Clostridium perfringens theta-toxin on erythrocyte membranes. A fluorescence energy transfer study. Journal of Biological Chemistry, 266(11), 6936-6941.
20. Piewngam, P., Zheng, Y., Nguyen, T. H., Dickey, S. W., Joc, H. S., Villaruz, A. E., . . . & Otto, (2018). Pathogen elimination by probiotic Bacillus via signalling interference. Nature, 562(7728), 532-537.

1. A composition for treating diseases or disorders caused by C. perfringens comprising Bacillus subtilis spores and its culture supernatant.

2. The composition of claim 1 comprising fengycin.

3. The composition of claim 1 comprising at least 2 ppm fengycin.

4. The composition of claim 1 wherein the strain of Bacillus subtilis is PB6.

5. A composition for treating diseases or disorders caused by C. perfringens comprising fengycin.

6. A feed composition for treating diseases or disorders caused by C. perfringens comprising fengycin.

7. A method of treating diseases or disorders caused by C. perfringens comprising the step of administering to an animal a composition comprising fengycin.

8. The method of claim 7 wherein the composition comprises Bacillus subtilis PB6 spores and its culture supernatant.

9. The method of claim 7 wherein the composition is administered to the animal through the animal's feed.

10. The method of claim 7 wherein the composition is administered to the animal through the animal's water.

11. The method of claim 7 wherein the composition is administered to the animal in a pharmaceutically acceptable carrier.

12. The method of claim 11 wherein the pharmaceutically acceptable carrier is selected from the group consisting of a tablet, capsule, liquid, suspension, and a suppository.

13. The method of claim 7 wherein the composition is administered to a chicken for treatment of avian necrotic enteritis.

14. The method of claim 13 wherein the composition is administered to the chicken in a dose of fengycin ranging from about 0.001 g/ton of feed to about 0.100 g/ton of feed.

15. The method of claim 11 wherein the composition is administered to the animal in a dose of from about 0.1 to about 100 μg/day.

16. A method of manufacturing a composition for treating diseases or disorders caused by C. perfringens comprising combining Bacillus subtilis PB6 spores and its culture supernatant with a carrier.

17. The method of claim 16 wherein the carrier is animal feed.

18. The method of claim 16 wherein the carrier is a pharmaceutically acceptable carrier.

19. The method of claim 16 wherein the pharmaceutically acceptable carrier is selected from the group consisting of a tablet, capsule, liquid, suspension, and a suppository.

20. The method of claim 16 wherein the carrier is an aqueous solution or water.
